(19) United States
(12) Patent Application Publication
(10) Pub. No.: US 2025/0049864 A1
(43) Pub. Date: Feb. 13, 2025

(54) PROBIOTIC AND USES THEREOF

(71) Applicants: INSTITUT NATIONAL DE RECHERCHE POUR L'AGRICULTURE, L'ALIMENTATION ET L'ENVIRONNEMENT, Paris (FR); INSTITUT DES SCIENCES ET INDUSTRIES DU VIVANT ET DE L'ENVIRONNEMENT, Palaiseau (FR); UNIVERSITÉ CLERMONT AUVERGNE, Clermont-Ferrand Cedex 1 (FR); ASSISTANCE PUBLIQUE - HOPITAUX DE PARIS, Paris (FR); UNIVERSITÉ DE PARIS CITÉ, Paris (FR); UNIVERSITE PARIS-SACLAY, Gif-sur-Yvette (FR)

(72) Inventors: Muriel THOMAS, Igny (FR); Isabelle SAVARY-AUZELOUX, Clermont-Ferrand (FR); Marianne JARZAGUET, Loubeyrat (FR); Camile MAYEUR, Beaune (FR); Stéphanie BORNES, Teissieres-Les-Boulies (FR); Muriel GIRON, Le Crest (FR); Dominique DARDEVET, Perignat Les Sarlieves (FR); Françoise JOLY, Paris (FR); Christophe CHASSARD, Aurillac (FR)

(21) Appl. No.: 18/567,852

(22) PCT Filed: Jun. 8, 2022
(86) PCT No.: PCT/EP2022/065569

§ 371 Date: Oct. 2, 2024

(30) Foreign Application Priority Data
Jun. 9, 2021 (FR) FR2106084

(51) Int. Cl. A61K 35/747; A61P 3/04; A61P 3/10; A61P 21/00

(52) U.S. Cl. CPC A61K 35/747; A61P 3/04; A61P 3/10; A61P 21/00

(57) ABSTRACT

The present invention relates to a composition comprising bacteria of the strain L. casei SGC-K deposited at the National Collection of Cultures of Microorganisms in the Institut Pasteur, Mar. 8, 2021, under no. CNCM 1-5663. The invention also relates to the nutritional and therapeutic uses of said composition.

### TECHNICAL FIELD

The present description relates to the field of the treatment of pathological or non-pathological situations of malnutrition leading to a loss of muscle mass and/or involving the presence of dysregulation of insulin sensitivity.

### PRIOR ART

Malnutrition affects an increasing number of humans, including in developed countries. According to a report by the Société Francophone, Nutrition Clinique et Métabolisme [Francophone Society of Clinical Nutrition and Metabolism], a prevalence of malnutrition ranging from 5% to 10% of the population can be estimated. It is greater in the elderly French population, in which the proportion of people aged over 75 will reach one in five people in 2050 (data from INSEE), including two million individuals with dementia (projection from the Paquid cohort, 2003). There is thus an aging population that is malnourished and highly sarcopenic, generally dependent, living in institutions, and in a state of chronic undernutrition (Buckinx et al., Archives of Public Health—Archives belges de santé publique [Belgian archives of public health], Vol. 73 (1): 19).

As is known, malnutrition results from an imbalance between (i) the nutritional needs of a subject and (ii) the nutritional intake of said subject. The nutritional needs of a healthy subject, at a given moment, can vary for example according to the intensity of the physical exercise performed daily, or else by the need to ensure appropriate physical growth. The nutritional needs of a sick subject, at a given moment, can also for example be increased due to the need to counteract a pathological condition, which can eventually lead to a state of cachexia, as the case may be in a subject affected by cancer. Thus, whether it is a healthy subject or a subject affected by a pathological condition, a state of malnutrition is remedied by providing said subject with nutrients of which the quantity and/or quality are adapted to the nutritional objective or the medical purpose being pursued.

Thus, malnutrition is defined in the prior art as being a “state of an organism in nutritional imbalance” (see “Diagnostic de la dénutrition de l′enfant et de l′adulte—Méthode Recommandations pour la pratique clinique” [“Diagnosis of malnutrition in children and adults—Method Recommendations for clinical practice”], November 2019, Haute Autorité de Santé [French National Authority for Health] and Fédération Française de Nutrition [French Society of Nutrition]), said imbalance being characterized by a negative energy and/or protein balance. This definition encompasses a plurality of situations that can lead to a state of malnutrition, such as an isolated nutrient intake deficiency, an increase in expenditure or losses leading to an imbalance. The imbalance inherent to malnutrition leads to deleterious effects on the body, accompanied by measurable changes in bodily functions and/or body composition, it being possible for these changes to be associated with worsening of the development of diseases from which the subject may be affected.

There are two main forms of malnutrition, each at the end of the same pathophysiological continuum. Both forms can coexist. Clinical criteria have been specified for these two forms. At one end, marasmus, without edema, by isolated intake deficiency, the translation of which is mainly anthropometric (weight loss and/or low BMI (Body Mass Index)), for example in the case of non-decompensated anorexia nervosa. At the other end, the hypercatabolic form with edema. This form combines an intake deficiency and metabolic stress, causing protein loss and a loss of muscle mass and function. The translation of metabolic stress is mainly biological (inflammatory syndrome and/or decreased albumin).

The consequences of malnutrition are numerous and represent a public health issue: lengthening of hospital stays, increase in risks of nosocomial infection, falls, fractures or becoming dependent, immune deficiency, depression, worsening of chronic diseases with the ultimate consequence of an increase in mortality. The etiological situations promoting malnutrition are the situations in which there is a reduction in food intake, an intestinal absorption disorder and/or a situation of protein hypercatabolismwith or without inflammatory syndrome which is called a situation of aggression. The diagnosis of malnutrition is therefore based on the presence of an etiological criterion and the presence of at least one phenotypic criterion. Thus, the new HAS 2019 criteria have issued the following criteria for subjects between 18 and 70 years old:
loss of weight ≥5% in 1 month or ≥10% in 6 months or ≥10% with respect to the usual weight before the onset of illness
subjects having a body mass index (BMI) of less than 18.5 kg/m²
quantified reduction in muscle mass and/or function.

For subjects aged over 70 years, the diagnostic criteria are in the process of being reevaluated by the HAS as they are now considered to be too out-of-date. However, they will also take into account BMI (Body Mass Index), weight loss kinetics and muscle function/strength. It is important to note that an elderly subject is at very high risk of being malnourished due to the higher frequency of comorbidities and therefore of hypercatabolic state, oral problems and polypharmacy that may alter food intake. Reduction in food intake can also be promoted by an inadequate social context. Social and/or familial isolation, lack of help with organizing shopping and preparing meals and insufficient financial resources have an impact on the quality of meals. Reduction in the mental (depression, dementia, etc.) and physical capacities of an elderly person lead to a loss of autonomy for activities of daily life and can participate in the appearance of malnutrition which itself will aggravate the physiological state of the patient.

Nowadays, the evaluation of muscle mass and/or muscle strength/function have become diagnostic criteria since numerous studies have shown the importance of the consequences of muscle wasting or sarcopenia in the prognosis of patients. Sarcopenia (word from Greek which can be translated as “lack of flesh”), initially defined by a loss of skeletal muscle mass, is currently characterized by a loss of muscle mass associated with functional deterioration. It constitutes a factor of severity of malnutrition.

The majority of malnourished people are found in health establishments. Thus, in France, in a short-stay establishment, the proportion of malnourished people is respectively (i) 20% for children, (ii) 45% for adults and (iii) 60% for elderly people.

However, malnutrition also concerns human populations that are not directly affected by malnutrition caused by the impossibility of access to a sufficient amount of food. These are, as mentioned above, in particular elderly people but also people affected by a pathological condition leading to malnutrition, such as cancer patients undergoing therapeutic treatment.

Malnutrition particularly affects cancer patients affected by cachexia, which is accompanied by a reduction in organ function, an impairment of the immune state and a decrease in muscle strength.

There also exist situations of malnutrition in obese people who are following a diet that may lead to a dietary deficiency. In obese people, a situation of stress, for example resulting from a surgical operation, particularly obesity surgery, can lead to malnutrition while the body mass index is greater than 30. Some overweight or obese people are affected by an increased susceptibility to developing diabetes, for example diabetes involving insulin resistance, or cardiovascular pathological conditions, and exhibit an increased loss of muscle mass and, as the case may be, an alteration in their body composition, which complicates the determination of appropriate treatments for obesity by implementing low-calorie diets (Barazzoni et al. 2018, Obesity Facts, Vol. 11 (4): 294-305). In obese people restricted to a low-calorie diet, it is known that the loss of fat mass can be accompanied by a loss of muscle mass. Salles et al. (2020, Diabetol Metab Syndr, Vol. 12:98) report the use of certain specific strains of L. casei. The strain L. casei CCFM419 is described as inducing a reduction in the HOMA-IR value.

A situation of malnutrition can also occur in the case of chronic digestive pathological conditions (such as inflammatory bowel diseases or short bowel syndrome), kidney failure or else chronic bronchopathy or respiratory infections. Malnutrition can also be the result of a poor oral state, a swallowing disorder, or else a drastic diet.

Malnutrition can also occur in pregnant women.

Athletic subjects have considerable protein and energy requirements in order to support intense physical activity. This increased energy requirement, if not accompanied by an appropriate diet, can lead to a loss of muscle mass. This decrease in muscle mass, resulting from intense physical exercise, can be compensated by ingestion of food supplements, in particular by oral intake of protein supplements, intake of specific amino acids such as leucine etc., the ingestion of these food supplements being aimed at preserving the lean body mass. Athletic subjects can anticipate these protein and energy requirements by ingestion of supplements during and/or after the exercise, with the aim of maintaining and/or replenishing nutritional reserves.

Thus, whether it is frail elderly people, patients suffering from cancer or obese individuals subjected to a low-calorie diet, the problem of muscle wasting is a key element, in a context of food intake below requirements, both in terms of energy and protein, which is accompanied by a loss of fat mass but also by a loss of muscle mass.

The therapeutic care of people affected by malnutrition is based on a plurality of principles, including (i) an assessment of the nutritional state, as quickly as possible, and (ii) the choice of a nutritional approach according to the spontaneous food intake of the malnourished person and according to the severity of the malnutrition. The therapeutic care of malnourished people can include the oral intake of nutritional supplements, intended in particular to reduce the concomitant loss of muscle mass.

Among the nutritional supplements which have been studied in the prior art, mention may be made of nutritional supplements comprising probiotics. Several trials have been carried out on malnourished children. Reference may for example be made to a randomized clinical trial (PRONUT study) aimed at evaluating the clinical and nutritional efficiency of a food mixture comprising four probiotic bacteria (Pediococcus pentosaceus, Leuconostoc mesenteroides, Lactobacillus paracasei, Lactobacillus plantarum) in combination with four types of prebiotics (inulin, pectin, oat bran, resistant starch) in Malawian children aged 5 to 168 months (Kerac et al., 2009, Lancet, Vol. 374:136-144). No significant improvement in nutritional state, mortality, weight gain, recovery time or the prevalence of clinical symptoms such as diarrhea, fever or respiratory problems was observed. On the contrary, another, non-randomized, clinical trial demonstrated that supplementation with probiotics increased the weight and height of Indian children with stunted growth (Saran et al., 2002, Nutrition, Vol. 18:393-396). Reference may also be made to the works of Matsuo et al. (2013, Internal Society of Exercise and Immunology, 11th Symposium, Newcastle, Australia) which showed, in a mouse model of skeletal muscle damaged by injection of a cardiotoxin, that the oral administration of L. casei caused recovery of muscle mass and regeneration of muscle tissue. Ni et al. (2019, Mol Nutr Food Res, Vol. 63:1900603) showed that supplementation of young or old mice with L. casei or Bifidobacterium longum increased muscle function and strength. Mention may also be made of patent application US 2017/080034, which relates to the use of compositions comprising Lactobacillus to stimulate growth in malnourished subjects.

With regard to the designation of certain probiotics, and in particular of bacteria of the genus Lactobacillus, it is important to note the recent changes made to their taxonomic classification, as reported in the article by Zheng et al. (2020, Int. J Syst Evol Microbiol, Vol. 70:2782-2858). Thus, certain probiotics of the genus Lactobacillus that are documented in the literature published prior to these taxonomic changes are now designated according to the new nomenclature in force.

By way of illustration, certain changes in the designation of Lactobacillus species made according to the new classification in force are described in Table 1 below.

TABLE 1
Correspondence between the old and new taxonomic designations
of certain species of Lactobacillus.

| Old designation | New designation |
|---|---|
| Lactobacillus acidophilus | Lactobacillus acidophilus |
| Lactobacillus gasseri | Lactobacillus gasseri |
| Lactobacillus helveticus | Lactobacillus helveticus |
| Bulgaricus lactobacillo | Lactobacillus delbrueckii subsp. bulgaricus |
| Lactobacillus brevis | Levilactobacillus brevis |
| Lactobacillus casei | Lacticaseibacillus casei |
| Lactobacillus fermentum | Limasilactobacillus fermentum |
| Lactobacillus paracasei | Lacticaseibacillus paracasei |
| Lactobacillus plantarum | Lactiplantibacillus plantarum |
| Lactobacillus reuteri | Limosilactobacillus reuteri |
| Lactobacillus rhamnosus | Lacticaseibacillus rhamnasus |
| Lactobacillus salivarius | Ligilactobacillus salivairius |

The correspondence between taxonomic designations according to the old and new nomenclature can also be found on the internet at the following address: http://lactobacillus.ualberta.ca/.

With regard to the treatment of malnutrition concomitant with cancer, mention may be made of the study by Bindels et al. (2012, PloS one, Vol. 7 (6): e37971) which studied the intestinal microbiota in a model of cachectic mice affected by acute leukemia. The treatment of these mice with oral supplementation containing Lactobacillus reuteri led to a decrease in markers of muscular atrophy in the gastrocnemius and tibialis anterior muscles. Furthermore, Varian et al. (2016, Oncotarget, Vol. 7 (11): 11803-11816) showed, in a mouse model of cachexia, that supplementation with a commensal bacterium, L. reuteri, reduced systemic indices of inflammation and inhibited cachexia.

Additionally, supplementation with certain probiotics, such as certain strains of Lactobacillus, of Bifidobacterium, of Clostridium or else of Akkermansia, Blautia, F. prausnitzii may provide beneficial changes in situations of insulin resistance (Izabel et al., 2020, Diabetol Metab Syndr, Vol. 12:98).

There remains a need for treatments of malnourished subjects, including elderly subjects, obese and/or diabetic subjects, subjects whose physical exercise has led to a loss of muscle mass or else cancer subjects affected by cachexia.

### SUMMARY OF THE INVENTION

The present description relates to a composition comprising bacteria of the strain L. casei SGC-K deposited with the Collection Nationale de Cultures de Microorganismes [French National Collection of Microorganism Cultures] of the Institut Pasteur on Mar. 8, 2021 under No. CNCM I-5663.

In certain embodiments, the bacteria of the strain L. casei SGC-K are combined with the bacteria of one or more other probiotic bacterial strains, including other probiotic bacterial strains of commensal origin.

The other probiotic bacterial strains may be chosen from the genera Bifidobacterium, Lactobacillus (and the new species described of the new genera described), Lactococcus, Enterococcus, Streptococcus, Kluyveromyces, Saccharomyces, Faecalibacterium, Blautia, Christensenella, Bacteroides, Eubacterium, Roseburia, Coprococcus and combinations thereof.

The other bacterial strains may be chosen from the species Bifidobacterium longum, Bifidobacterium lactis, Bifidobacterium breve, Bifidobacterium infantis, Bifidobacterium adolescentis, Lactobacillus acidophilus, Lactobacillus casei, Lactobacillus paracasei, Lactobacillus salivarius, Lactobacillus lactis, Lactobacillus rhamnosus, Lactobacillus johnsonii, Lactobacillus plantarum, Lactobacillus salivarius, Lactococcus lactis, Enterococcus faecium, Enterococcus faecalis, Saccharomyces cerevisiae, Saccharomyces boulardii, Faecalibacterium prausnitzii or mixtures thereof, preferably chosen from the group consisting of Bifidobacterium longum NCC3001 (ATCC BAA-999), Bifidobacterium longum NCC2705 (CNCM 1-2618), Bifidobacterium longum NCC490 (CNCM 1-2170), Bifidobacterium lactis NCC2818 (CNCM I-3446), Bifidobacterium breve strain A, Lactobacillus paracasei NCC2461 (CNCM 1-2116), Lactobacillus johnsonii NCC 533 NCC533 (CNCM 1-1225), Lactobacillus rhamnosus GG (ATCC53103), Lactobacillus rhamnosus NCC4007 (CGMCC 1. 3724), Enterococcus faecium SF 68 (NCC2768; NCIMB10415), and combinations thereof.

In certain embodiments, the composition also comprises one or more prebiotics.

The present application also relates to the use of a composition as defined in the present description, for maintaining or increasing muscle mass and/or function in a subject in need thereof, in particular in a malnourished subject, in an elderly subject, in particular a malnourished elderly subject, and in a subject performing intense physical exercise.

The present application also relates to a composition as defined in the present description for use thereof as a medicament.

The present application also relates to a composition as defined in the present description, for use thereof for the treatment of the loss of muscle mass and/or function in a subject in need thereof, in particular in a subject chosen from overweight subjects and obese subjects subjected to a diet, cancer subjects affected by sarcopenia, diabetic subjects subjected to a diet.

The present application also relates to a composition as defined in the present description, for use thereof for reducing insulin resistance in a subject in need thereof, in particular in a diabetic subject, and more particularly a subject affected by type 2 diabetes.

The present description also relates to the strain L. casei SGC-K deposited with the Collection Nationale de Cultures de Microorganismes [French National Collection of Microorganism Cultures] of the Institut Pasteur on Mar. 8, 2021 under No. CNCM I-5663.

### BRIEF DESCRIPTION OF THE DRAWINGS

FIG. 1 represents the loss of lean body mass at D 58, at the end of the 60-day test protocol. On the abscissa, the groups of animals tested, respectively (i) “AL” control group of animals fed ad libitum in white, (ii) “R”, group of animals with restricted nutrition having received the control PBS buffer without probiotic in black, (iii) “R+SGC-K”: group of animals with restricted nutrition having received the probiotic L. casei SGC-K in the PBS buffer in gray. On the ordinate, the loss of lean body mass, expressed in grams. A change of letter (a, b, c) indicates a significant difference between the groups. Thus, the difference is statistically significant (p<0.05-n=59) respectively, (i) between groups “a” and “b”, (ii) between groups “a” and “c” and (ii) between groups “b” and “c”.

FIG. 2 represents the insulin sensitivity state of the animals illustrated by the results of the HOMA-IR calculation. On the ordinate, each group of two bars illustrates the results at DO (black left bar) and at D60 (striped right bar), respectively for the following groups of animals (n=53): (i) “AL” control group of animals fed ad libitum, (ii) “R”, group of animals with restricted nutrition having received the control PBS buffer without probiotic, (iii) “R+SGC-K”: group of animals with restricted nutrition having received the probiotic L. casei SGC-K in the PBS buffer.

FIG. 3 represents the insulin sensitivity state of the animals illustrated by the glycemia results following an oral glucose test (OGTT-Oral glucose tolerance test) after 60 days of restriction and treatment with SGC-K probiotics. The upper curve (white circles) illustrates the results obtained for the “AL” group of animals, which is the control group of animals fed ad libitum. The middle curve (black circles) illustrates the results obtained for the “R” group of animals, which is the group of animals with restricted nutrition having received the control PBS buffer. The lower curve (gray circles) illustrates the results obtained for the “R+SGC-K” group of animals, which is the group of animals with restricted nutrition having received the probiotic L. casei SGC-K in the PBS buffer. A change of letter (a, b, c) indicates a significant difference (p<0.05-n=53).

FIG. 4 represents the mass (in grams) of the sum of the hind paw muscles of the rats obtained after dissection of the paw (gastrocnemius, soleus, tibialis anterior and extensor digitorum longus muscles) following 30 days of dietary restriction and supplementation with the probiotic L. casei SGC-K. On the abscissa, the groups of animals tested (n=61), respectively (i) “AL” control group of animals fed ad libitum in white, (ii) “R”, group of animals with restricted nutrition having received the control PBS buffer without probiotic in black, (iii) “R+SGC-K”: group of animals with restricted nutrition having received the probiotic L. casei SGC-K in the PBS buffer in gray. On the ordinate, the muscle mass, expressed in grams. The difference is statistically significant (p<0.05-n=61) between groups R and R+SCG-K. The difference is not significant between groups AL and R (P=0.21) and between groups AL and R+SCG-K (P=0.72).

FIG. 5 represents the level of phosphorylation of the protein S6 in an aged rat model following 30 days of dietary restriction and supplementation with the probiotic L. casei SGC-K, respectively (a) in animals fed ad libitum, (b) in animals subjected to dietary restriction and (ab) in animals subjected to dietary restriction and supplemented with strain SGCK. On the ordinate, the values of the phosphorylated protein S6 (p-S6)/non-phosphorylated protein S6 (S6) ratio, i.e. the “p-S6/S6” ratio. The results are those of the ratio of the densities of p-S6 and of S6 in a Western blot assay.

FIG. 6 represents the level of phosphorylation of the elongation factor clF2a in an aged rat model following 30 days of dietary restriction and supplementation with the probiotic L. casei SGC-K, respectively (a) in animals fed ad libitum, (b) in animals subjected to dietary restriction and (c) in animals subjected to dietary restriction and supplemented with strain SGCK. On the ordinate, the values of the phosphorylated protein elF2a (p-elF2a)/non-phosphorylated protein elF2a (elF2a) ratio, i.e. the “p-elF2a/elF2a” ratio. The results are those of the ratio of the densities of p-elF2a and of elF2a in a Western blot assay.

### DETAILED DESCRIPTION

What has been identified according to the present description is a strain of L. casei which has a plurality of beneficial properties for the health of subjects affected by malnutrition.

The strain of L. casei according to the present description consists in the strain of L. casei SGC-K deposited on Mar. 8, 2021 with the Collection Nationale de Cultures de Microorganismes [French National Collection of Microorganism Cultures] of the Institut Pasteur under No. CNCM I-5663. This strain may also be referred to as “L. casei SGC-K” or “SGC-K” in the present description.

As shown in the examples, on a malnourished subject model, the oral administration of the strain L. casei SGC-K leads to a reduction in the loss of muscle mass in these subjects affected by malnutrition, in comparison with malnourished subjects that have not received the strain SGC-K. More particularly, the strain L. casei SGC-K leads to a reduction in the loss of lean muscle mass in these subjects affected by malnutrition, in comparison with malnourished subjects that have not received the strain SGC-K.

In addition, the results reported in the examples show that the oral administration of the strain L. casei SGC-K enables a significant decrease in glycemia in malnourished subjects, in comparison with subjects that have not received the strain SGC-K. These experimental results show that the administration of the strain SGC-K causes an insulin-sensitizing effect; this may be beneficial in subjects in need thereof, in particular in subjects affected by insulin resistance.

The results reported in the examples also show that the administration of the strain SGC-K induces stimulation of the expression of biomarkers involved in the intracellular signaling of insulin, such as phosphorylated protein S6 or else phosphorylated elongation factor elF2a, as visualized by an increase in the respective ratios p-S6/S6 and p-elF2a/elF2a, in which (1) “p-S6” denotes phosphorylated protein S6 and “S6” denotes non-phosphorylated protein S6 and (2) “p-elF2a” denotes phosphorylated elongation factor elF2a and “elF2a” denotes non-phosphorylated elongation factor elF2a.

According to the invention, protein “S6” means the well-known ribosomal protein S6, which is a component of the 40S subunit of the ribosome involved in translation.

According to the invention, elongation factor “elF2a” means the well-known protein which is a translation initiation factor, necessary for protein synthesis.

As is known, the occurrence of insulin resistance can be a marker of metabolic disorders, such as metabolic syndrome, which metabolic disorders represent a risk factor for developing pathological conditions. Thus, these metabolic disorders can for example lead to the occurrence of type 2 diabetes, promote obesity, and be an element predisposing to cardiovascular pathological conditions such as coronary risk, certain non-alcoholic liver diseases (hepatic steatosis), hypertension; polycystic ovary syndrome (PCOS), chronic intestinal pathological conditions, chronic intestinal kidney pathological conditions or else metabolic syndrome.

As is known, metabolic syndrome is defined by the presence of values considered to be abnormal for at least three of the following five criteria: (i) waist size, (ii) triglyceride level, (iii) HDL cholesterol level, (iv) blood pressure and (v) fasting glycemia value. The values considered to be abnormal may differ depending on the sex of the subject, it also being possible for these values to be ethnocentered (see for example Alberti et al., 2005, “The metabolic syndrome—a new worldwide definition”; doi: 10.1016/S0140-6736 (05) 67402-8). By way of illustration, the occurrence of metabolic syndrome may be diagnosed if at least three criteria are met, among the following five criteria: (i) a waist size of greater than 102 cm in men and greater than 88 cm in women, (ii) a triglyceride level of greater than 150 mg/dL (1.7 mmol/L), or the following of a specific treatment against an excess of triglycerides, (iii) an HDL cholesterol level of less than or equal to 40 mg/dl (1.03 mmol/L) in men or less than or equal to 50 mg/dL (1.29 mmol/L) in women, or the following of a treatment against this lipid abnormality, (iv) a systolic blood pressure value of greater than 130 mmHg or diastolic blood pressure value of greater than 85 mmHg, or the following of a treatment against hypertension, or (v) a fasting glycemia value of greater than or equal to 100 mg/dL (5.6 mmol/L) or the presence of type 2 diabetes.

As is also known, inflammatory situations are linked to insulin resistance. The administration of the strain SGC-K is therefore also useful for the prevention or the treatment of inflammatory pathological conditions such as (i) irritable bowel syndrome or disease (or “IBS/IBD”), (ii) chronic obstructive pulmonary disease (or “COPD”), or else (iii) other pathological conditions linked to chronic inflammation, including chronic intestinal or kidney inflammation, such as long-term inflammation caused by a viral infection such as an infection by a SARS-COV-2 virus.

The present description relates to the strain L. casei SGC-K deposited with the Collection Nationale de Cultures de Microorganismes [French National Collection of Microorganism Cultures] of the Institut Pasteur on Mar. 8, 2021 under No. CNCM I-5663 (also called strain “L. casei SGC-K” or “SGC-K”).

For the purposes of the present description, the strain SGC-K is a probiotic.

As used herein, the term “probiotic” denotes live microorganisms which, included in a sufficient amount, have a positive effect on physiology, comfort and well-being.

### A. Compositions

The present description also relates to a composition comprising the strain SGC-K.

As will be apparent in the description, a composition comprising the strain SGC-K is primarily intended for malnourished subjects, including malnourished subjects affected by type 2 diabetes.

According to the description, “malnourished subject” means a subject with a state of malnutrition. The state of malnutrition is well known to those skilled in the art. The characteristics of a state of malnutrition and its consequences are documented in the introduction to the present description (see also “Diagnostic de la dénutrition de l′enfant et de l′adulte—Méthode Recommandations pour la pratique clinique” [“Diagnosis of malnutrition in children and adults-Method Recommendations for clinical practice”], November 2019, Haute Autorité de Santé [French National Authority for Health] and Fédération Française de Nutrition [French Society of Nutrition], pages 11-12). Malnourished subjects include humans and non-human animals, including non-human mammals. Malnourished subjects therefore include livestock, farm animals and pets. Malnourished subjects include farmyard animals (such as chickens, turkeys, ducks, geese, pigeons, quails, pheasants, ostriches, emus, rheas and guinea fowl). Malnourished subjects also include grazing animals, such as equines (such as horses, donkeys, mules, hinnies), porcines, bovines (such as cows, buffalo, zebus, bison, aurochs, yaks), ovines, caprines, camelids (such as camels, dromedaries, llamas, alpacas) and cervids (such as reindeer, deer). Malnourished subjects also include pets such as felines and canines.

Malnourished subjects include humans, including malnourished children and elderly people.

The present description relates to a nutritional composition comprising the strain SGC-K, which is intended for malnourished subjects, affected by a loss of muscle mass, said subjects not having any pathological condition toward which said nutritional composition has a prophylactic and/or therapeutic effect.

By way of example, an athletic subject in which it is necessary to maintain or increase muscle mass, who does not have any pathological condition and only requires a contribution of nutritional nature. According to another example, the composition according to the present description may be a nutritional composition intended for malnourished elderly people, for overweight people that are malnourished for example because they are following a diet.

For the purposes of the present description, “elderly subject” means a human subject or a non-human mammalian subject, including a pet such as a dog or cat, who exhibits signs of senescence such as an alteration of metabolic functions (for example absorption, digestion, excretion), locomotor difficulties and reduced resistance to external aggressions.

“Elderly subject”, or “elderly person”, in the case of a human, means a subject aged 65 years or over.

“Elderly subject”, in the case of a canine, in particular a dog, means a subject aged (i) over 12 years for a canine, in particular a dog, of small size or (ii) over 9 years for a canine, in particular a dog, of medium size or (iii) over 7 years for a canine, in particular a dog, of large size.

“Elderly subject”, in the case of a feline, in particular a cat, means a subject aged over 13 years.

The present description also relates to a pharmaceutical composition comprising the strain SGC-K, which is intended for subjects having, or likely to have, a pathological condition for which said composition has a prophylactic or therapeutic effect. By way of example, a composition according to the present description consists in a pharmaceutical composition when it is intended to prevent or treat a situation of insulin resistance in a subject, such as for example in a subject affected by type 2 diabetes.

However, unless specifically stated, a composition comprising the strain SGC-K does not differ in its general characteristics set out in the description depending on whether it is a nutritional composition or a therapeutic composition. Thus, essentially, a pharmaceutical composition is distinguished from a nutritional composition by the fact that the pharmaceutical composition has, in the subject to which it is administered, a preventive effect and/or a treatment effect of a disease, specifically an insulin-sensitizing effect in an insulin-resistant subject.

In certain embodiments of a composition according to the description, the strain SGC-K is used as the only bacterial strain present in the composition.

In certain other embodiments of a composition according to the description, the bacteria of the strain SGC-K is combined with bacteria of one or more other probiotic bacterial strains, which includes commensal probiotic bacterial strains.

Non-limiting examples of probiotics include the strains of bacteria belonging to the following genera: Bifidobacterium, Lactobacillus, Lactococcus, Enterococcus, Streptococcus, Kluyveromyces, Saccharomyces, Candida and combinations thereof.

The probiotics may be selected from the group consisting of the following bacterial species: Bifidobacterium longum, Bifidobacterium lactis, Bifidobacterium breve, Bifidobacterium infantis, Bifidobacterium adolescentis, Lactobacillus acidophilus, Lactobacillus casei (now Lacticaseibacillus casei), Lactobacillus paracasei (now Lacticaseibacillus paracasei), Lactobacillus salivarius (now Ligilactobacillus salivarius), Lactobacillus lactis (now Lactobacillus delbrueckii subsp. Lactis), Lactobacillus rhamnosus (now Lacticaseibacillus rhamnosus), Lactobacillus johnsonii, Lactobacillus plantarum (now Lactiplantibacillus plantarum subsp. Plantarum), Lactococcus lactis, Enterococcus faecium, Enterococcus faecalis, Saccharomyces cerevisiae, Saccharomyces boulardii or mixtures thereof, preferably selected from the group consisting of Bifidobacterium longum NCC3001 (ATCC BAA-999), Bifidobacterium longum NCC2705 (CNCM 1-2618), Bifidobacterium longum NCC490 (CNCM 1-2170), Bifidobacterium lactis NCC2818 (CNCM I-3446), Bifidobacterium breve strain A, Lactobacillus paracasei NCC2461 (CNCM 1-2116), Lactobacillus johnsonii NCC533 (CNCM 1-1225), Lactobacillus rhamnosus GG (ATCC53103), Lactobacillus rhamnosus NCC4007 (CGMCC 1. 3724), Enterococcus faecium SF 68 (NCC2768; NCIMB10415), Faecalibacterium prausnitzii and combinations thereof.

In certain embodiments, the composition also comprises one or more prebiotics. The term “prebiotic” is used in its conventional sense in the prior art. Prebiotics consist in food substances that promote the growth of probiotic bacteria, including the bacteria included in the microbiota.

Non-limiting examples of prebiotics comprise: oligosaccharides which may contain fructose, galactose, mannose; dietary fibers, in particular fermentable fibers, soybean fibers; inulin; and combinations thereof. Preferred prebiotics are fructooligosaccharides (FOS), galactooligosaccharides (GOS), isomaltooligosaccharides (IMO), xylooligosaccharides (XOS), arabinoxylooligosaccharides (AXOS), mannan oligosaccharides (MOS), soybean oligosaccharides, glycosylsucrose (GS), lactosucrose (LS), lactulose (LA), palatinose oligosaccharides (PAO), maltooligosaccharides, resistant starches, gums and/or hydrolyzates thereof, pectins and/or hydrolyzates thereof, or combinations thereof.

In certain embodiments, the composition also comprises one or more vitamins. The vitamins may be folic acid, vitamin B12 and vitamin B6, in particular folic acid and vitamin B12, in particular folic acid. In certain embodiments, the composition comprises one or more vitamins that are liposoluble, for example one or more vitamins among vitamin A, vitamin D, vitamin E and vitamin K.

In certain embodiments, the composition comprises one or more polyphenols, such as flavanols, flavanones, flavonols, hydroxycinnamic acids and anthocyanins.

In certain embodiments, the composition also comprises one or more minerals. The minerals may be selected from sodium, potassium, chloride, calcium, phosphate, magnesium, iron, zinc, copper, selenium, manganese, fluorine, iodine, chromium or molybdenum. The minerals are generally added in salt form. The minerals may be added alone or in combination.

In certain embodiments, a composition according to the present description generally comprises carriers or vehicles. “Carriers” or “vehicles” denote materials suitable for administration and comprise any material known in the prior art, such as, for example, any liquid, gel, solvent, liquid diluent, solubilizing agent or other, which is non-toxic and which does not interact with the components of the composition in a deleterious manner. Examples of nutritionally acceptable carriers comprise, for example, water, saline solutions, alcohols, silicones, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroleum fatty acid esters, hydroxymethylcellulose, polyvinylpyrrolidone, etc.

In certain embodiments, the composition also comprises any other ingredient or excipient known to be employed in the type of composition in question. Non-limiting examples of such ingredients comprise: proteins, amino acids, carbohydrates, oligosaccharides, lipids, nucleotides, nucleosides, other vitamins, minerals and other micronutrients.

In certain embodiments, the composition contains a carbohydrate source, for example in the form of prebiotics, or of prebiotics when they are present in the composition. Use may be made of any carbohydrate source usually found in infant formulas, such as lactose, sucrose, maltodextrin, starch and mixtures thereof, although the preferred carbohydrate source is lactose.

In certain embodiments, a composition according to the present description consists in a nutritional composition.

In certain embodiments, the nutritional composition is chosen from complete food compositions, food supplements, nutraceutical compositions and others. The composition of the present description may be used as food ingredient and/or animal feed ingredient. The food ingredient may be in the form of a solution or a solid, depending on the use and/or the mode of application and/or the mode of administration. As used herein, the term “food” refers to liquid (i.e. drinks), solid or semi-solid dietary compositions, in particular total food compositions (food replacement), which do not require additional intake of nutrients or food supplement compositions. Food supplement compositions do not completely replace the intake of nutrients by other means. As used in the present description, the term “food ingredient” encompasses a formulation that is or can be added to functional foods or to foodstuffs as a food supplement. “Functional” or “nutraceutical” or “nutritional food” means a food material that contains ingredients having effects that are beneficial to health or capable of improving physiological functions. “Food supplement” means a food material intended to supplement a normal diet. A food supplement is a concentrated source of nutrients or other substances having a nutritional or physiological effect, when they are taken alone or in combination in small amounts. According to the present description, “functional food” is used to denote food materials and the corresponding products to which importance is attributed not only because of their nutritional and gustative value but also because of the presence of ingredients having beneficial physiological effects.

In certain embodiments, the composition is a fermented dairy product or a milk-based product, which is preferably administered or ingested orally one or more times per day. Fermented dairy products include milk-based products, such as (but not limited thereto) desserts, yogurts, yogurt drinks, fromage blanc, kefir, fermented milk-based drinks, buttermilk, vinaigrettes, low-fat spreads, fromage frais, soy-based drinks, ice cream, etc.

As a variant, in certain embodiments, the nutritional and/or nutritional supplement compositions may be non-dairy or non-fermented dairy products. Non-fermented dairy products may include ice creams, nutrition bars and seasonings, and others. Non-dairy products may include powdered drinks and nutrition bars, etc. The products may be produced using known methods, such as the addition of an effective amount of an SGC-K bacterium, or of a combination of bacteria including the strain SGC-K, to a food base, such as skim milk or milk or a milk-based composition, and carrying out a fermentation according to any known technique. In certain embodiments, the composition is a drink which may be a functional drink or a therapeutic drink, a thirst-quenching drink or a conventional drink. By way of example, the composition according to the present description may be used as an ingredient for carbonated drinks, a fruit juice or a drink comprising whey proteins, teas, cocoa drinks, milk drinks, yogurts including drinking yogurts, cheeses, ice creams, popsicles and desserts, confectionery, cookies, cakes and cake mixes, snacks, healthy foods and drinks, icings, acidified juice/soy drink, aseptic/reconstituted chocolate drink, mixes for bars, powdered drink preparations, calcium-enriched chocolate soy milk, calcium-enriched coffee drink.

In certain embodiments, the composition comprises any other ingredient or excipient known to be employed in the type of composition in question. Non-limiting examples of such ingredients comprise: proteins, amino acids, carbohydrates, oligosaccharides, lipids, prebiotics or probiotics, nucleotides, nucleosides, other vitamins, minerals and other micronutrients.

In certain other embodiments, bacteria of the strain SGC-K, where appropriate in combination with probiotic bacteria of one or more other strains, are administered to the subject in the form of a pharmaceutical composition, which could correspond to a product of the Live Biotherapeutic Product (LBP) type, reference: Front Med (Lausanne) Rouanet et al. 2020 Jun. 19; 7:237. doi: 10.3389/fmed.2020.00237. For example, the bacteria of interest may be combined with pharmaceutically acceptable excipients, and optionally sustained release matrices, such as biodegradable polymers, in order to form therapeutic compositions. The terms “pharmaceutically” or “pharmaceutically acceptable” denote molecular entities and compositions which do not produce an adverse reaction, allergic reaction or other reaction when they are administered to a mammal, in particular to a human, as appropriate.

A pharmaceutically acceptable carrier or excipient denotes a filler, a diluent, an encapsulating material or an auxiliary with a formulation that is non-toxic, solid, semi-solid or liquid, of any type. In the pharmaceutical compositions of the present description for oral, sublingual, subcutaneous, intramuscular, intravenous, transdermal, local or rectal administration, the active ingredient or the combination of active ingredients active ingredients, may be administered in a unit administration form, as a mixture with conventional pharmaceutical carriers, to animals and human beings. The suitable unit administration forms comprise oral administration forms such as tablets, gel capsules, powders, granules and oral suspensions or solutions, sublingual and buccal administration forms, aerosols, implants, subcutaneous, transdermal, topical, intraperitoneal, intramuscular, intravenous, subcutaneous, transdermal, intrathecal and intranasal administration forms and rectal administration forms. Typically, the pharmaceutical composition contain vehicles which are pharmaceutically acceptable for a formulation capable of being injected. These may in particular be isotonic, sterile, saline solutions (monosodium or disodium phosphate, sodium chloride, potassium chloride, calcium chloride or magnesium chloride and the like or mixtures of these salts), or dry compositions, in particular lyophilized compositions, which by addition, as appropriate, of sterilized water or of physiological saline enable the constitution of injectable solutions. The pharmaceutical forms suitable for injectable use comprise sterile aqueous solutions or dispersions; formulations comprising sesame oil, peanut oil or aqueous propylene glycol; and sterile powders for the extemporaneous preparation of sterile injectable solutions or dispersions. In any case, the form must be sterile and must be fluid to the point that it can be easily squeezed.

The pharmaceutical composition must be stable under the manufacture and storage conditions and must be preserved from the contaminating action of microorganisms, such as bacteria and fungi. Solutions comprising the compounds of the disclosure in the form of free base or of pharmacologically acceptable salts may be prepared in water mixed in a suitable manner with a surfactant, such as hydroxypropylcellulose. Dispersions may also be prepared in glycerol, liquid polyethylene glycols and mixtures thereof, as well as in oils. Under ordinary storage and use conditions, these preparations contain a preservative in order to prevent microorganism growth. The at least one SGC-K bacterium according to the present description may be formulated in a composition in a neutral form or in the form of a salt. The pharmaceutically acceptable salts comprise the acid addition salts (formed with the free amino groups of the protein), which addition salts are formed with inorganic acids such as, for example, hydrochloric acid or phosphoric acid, or organic acids such as acetic acid, oxalic acid, tartaric acid or mandelic acid, and the like. The salts formed with the free carboxyl groups may also be derived from inorganic bases such as, for example, sodium, potassium, ammonium, calcium or ferric hydroxides, and organic bases such as isopropylamine, trimethylamine, histidine, procaine and the like.

For the purposes of the present description, the term “therapeutically effective amount” is an equivalent expression which refers to the amount of a therapy (for example, a prophylactic or therapeutic agent) which is sufficient to reduce the severity and/or the duration of a disease, improve one or more of its symptoms, prevent the progression of a disease or cause the regression of a disease, or which is sufficient to result in the prevention of the development, the recurrence, the onset or the progression of a disease or of one or more of its symptoms, or reinforce or improve the prophylactic and/or therapeutic effect or effects of another therapy (for example, another therapeutic agent) useful for treating a disease. Typically, in a pharmaceutical composition according to the present description, the bacteria of the strain SGC-K are present in a sufficient amount to induce a reduction in the insulin resistance in the treated subject. The measurement of insulin resistance in a subject may be carried out according to any technique known to those skilled in the art. Preferably, the measurement of insulin resistance in a subject is carried out by calculating the HOMA-IR index, as illustrated in the examples. The HOMA-IR (for “HomeOstasis Model Assessment for Insulin Resistance”) method, which was developed from the mathematical modeling of the quantitative responses of the main organs of glucose metabolism. The value of the HOMA-IR index is obtained using a plasma value of insulin or C-peptide and fasting glycemia (Scheen, 2007, Thérapie [Therapies], Vol. 62:311-318). The occurrence and/or the level of insulin resistance can also be established by measuring fasting glycemia, by measuring fasting insulinemia or else by the OGTT test (for “Oral Glucose Tolerance Test”—See for example “Mesure de l′insulinorésistance et de la tolérance au glucose” [“Measurement of insulin resistance and glucose tolerance”], 2006, Haute Autorité de Santé [French National Authority for Health] (HAS), France).

In a composition according to the description, whether it is a nutritional composition or a food composition, where appropriate a food supplement, or whether it is a pharmaceutical composition, the bacterium or bacteria may be in various forms, for example in liquid form or in powder form. The bacterium or bacteria may be in lyophilized form.

The quantity of bacteria of the strain SGC-K that is provided to the subject may be variable, depending on the physiological state of said subject, and in particular depending on the level of nutrient intake imbalance relative to the nutrient requirement of said subject. The quantity of SGC-K bacteria which need to be provided to said subject may easily be adapted by those skilled in the art.

In preferred embodiments, whether the composition is a nutritional composition or a pharmaceutical composition, the composition comprises a quantity of bacteria of the strain SGC-K that is suitable for daily intake, preferably daily oral intake, of at least 10³ colony forming units (or “CFU”).

In these preferred embodiments, the daily intake, in particular the daily oral intake, of bacteria of the strain SGC-K is at most 10¹³ colony forming units (or “CFU”).

In embodiments in which the composition also comprises other probiotic bacteria, the quantity of these probiotic bacteria is determined by those skilled in the art based on their general knowledge. The quantity of these other probiotic bacteria may vary from 10³ to 10¹³ other probiotic bacteria.

In certain embodiments, a composition according to the present description generally comprises carriers or vehicles. “Carriers” or “vehicles” denote materials suitable for administration and comprise any material known in the prior art, such as, for example, any liquid, gel, solvent, liquid diluent, solubilizing agent or other, which is non-toxic and which does not interact with the components of the composition in a deleterious manner. Examples of nutritionally acceptable carriers comprise, for example, water, saline solutions, alcohols, silicones, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroleum fatty acid esters, hydroxymethylcellulose, polyvinylpyrrolidone, etc.

In other embodiments of a composition according to the present description, said composition is in the form of a pharmaceutical composition comprising one or more pharmaceutically acceptable excipients.

Such a pharmaceutical composition may be presented in the form of a pack comprising a plurality of dosage units.

The term “dosage unit” is used in its conventional sense in pharmacy (e.g. a pill, a capsule, a tablet, the contents of an ampoule, etc.).

### B. Uses

The present description relates to the nutritional uses and the therapeutic uses of a composition as defined in the present application.

The composition characteristics, including of a nutritional composition and of a therapeutic composition, including in various embodiments, are described in detail in the present description, which includes the quantities of active ingredient(s), especially the quantities of bacteria of the strain SGC-K comprised in these compositions.

The present description relates to the use of a composition as defined in the present application for maintaining or increasing muscle mass and/or muscle function in a subject in need thereof.

In numerous embodiments, a composition according to the present description consists in a nutritional composition, i.e. in a composition which is used for nutritional purposes for maintaining or increasing muscle mass and/or muscle function in a subject in need thereof. Such a use remains a nutritional use of non-therapeutic nature, including in certain sick subjects whose pathological condition affecting them is concomitant with a loss of muscle mass and/or of muscle function. Specifically, in these contexts, the provision of a nutritional composition according to the present description does not have the effect of preventing or treating the pathological condition affecting the subject, but has the effect of providing a nutritional benefit to said subject that is also receiving therapeutic treatment intended to treat said pathological condition.

In particular, the provision of a composition according to the present description to a subject not having a disorder associated with insulin resistance, whether this subject is or is not affected by a pathological condition, consists in a nutritional use of such a composition.

However, regulatory provisions may vary depending on the countries concerned, which means that a composition according to the present description, in its modes of use with nutritional effects described above, may, where appropriate, be considered as a medicament from a regulatory point of view which governs health law.

The present description relates to the use of a nutritional composition as defined in the present application for maintaining or increasing muscle mass and/or function in a subject in need thereof. Preferably, subjects in need of a nutritional composition as defined in the present application are selected from malnourished subjects, elderly subjects, in particular malnourished elderly subjects, and subjects performing intense physical exercise or else subjects having increased protein requirements.

In certain embodiments, said nutritional composition consists in a nutritional supplement composition as defined in the present description.

In certain embodiments, a nutritional composition in accordance with the present description, where appropriate in the form of a food supplement composition, is formulated for daily administration, for example for daily administration in one or two doses, for example at mealtimes.

In general, a nutritional composition according to the present description may be administered to the subject until the value of muscle mass and/or of muscle function reaches a predetermined level, indicative of normal muscle mass (see for example “Diagnostic de la dénutrition de l′enfant et de l′adulte-Méthode Recommandations pour la pratique clinique” [“Diagnosis of malnutrition in children and adults-Method Recommendations for clinical practice”], November 2019, Haute Autorité de Santé [French National Authority for Health] and Fédération Française de Nutrition [French Society of Nutrition], the table at the end of page 11). By way of illustration, the level of muscle mass and/or of muscle function can be determined with regard to at least one of the following criteria: (i) grip strength measured by a dynamometer, for example in kg, (ii) walking speed, for example measured in m/s, (iii) L3 muscle area index, for example measured in cm²/m², for example using a scanning device such as in MRI, (iv) muscle mass index, for example in kg/m², for example measured by impedance analysis, (v) fat-free mass index, for example in kg/m², for example measured by impedance analysis, or else (vi) appendicular muscle mass (DEXA), for example measured in kg/m². The measurement of muscle metabolic dynamics (which have the effect of regulating muscle mass and function) may also, in certain cases, be evaluated using more invasive techniques, for example techniques that make it possible to measure the rate of protein turnover by injecting amino acids labeled with stable isotopes.

In certain embodiments, a nutritional composition in accordance with the present description is administered to the subject chronically, for a long period of time, for example for several months or even for several years, for the purposes of maintaining the level of muscle mass and/or of muscle function at a predetermined value, indicative of the absence of normal muscle mass and/or of normal muscle function.

The present description also relates to medical uses of a composition as described in the present application.

The description relates to a composition as defined in the present application for use thereof as a medicament.

It relates to the use of a composition as defined in the present application for the manufacture of a medicament.

The description also relates to a composition as defined in the present application for use thereof for preventing or treating a loss of muscle mass in subjects in need thereof. Preferably, the subjects in need thereof are selected from overweight subjects and obese subjects, often insulin-resistant, and subjected to a low-calorie and/or low-carbohydrate diet, obese or non-obese subjects having undergone a bariatric surgical operation, who therefore have a sharp decrease in their overall energy intake but whose protein intake must be maintained to avoid muscle mass wasting, cancer subjects, in particular cancer subjects affected by cachexia, subjects treated with antibiotic therapy, subjects affected by a viral or bacterial infection, diabetic subjects subjected to a low-calorie and/or low-carbohydrate diet, or else in subjects in resuscitation who are fed enterally.

The description also relates to the use of a composition as defined in the present application for the manufacture of a medicament for preventing or treating a loss of muscle mass in subjects in need thereof. Preferably, the subjects in need thereof are selected from overweight subjects and obese subjects subjected to a low-calorie diet, obese or non-obese subjects having undergone a bariatric surgical operation, cancer subjects, in particular cancer subjects affected by sarcopenia, subjects treated with antibiotic therapy, subjects affected by a viral or bacterial infection, subjects affected by sarcopenia, diabetic subjects subjected to a low-calorie diet, or else in subjects in resuscitation who are fed enterally.

The description also relates to a method for preventing or treating a loss of muscle mass and function in subjects in need thereof comprising the administration to said subject of a composition as defined in the present application.

Thus, in certain embodiments, a composition as defined in the present application may be used preventively. The composition may for example be administered, preventively, to subjects at risk of developing a loss of muscle mass and function, which includes subjects affected by insulin resistance, for obese subjects following a treatment aimed at weight loss, for which a loss of muscle mass and/or a loss of muscle function has not occurred.

In general, a therapeutic composition according to the present description may be administered to the subject until the value of muscle mass and/or of muscle function reaches a predetermined level, indicative of an absence of sarcopenia.

In certain embodiments, a therapeutic composition in accordance with the present description is administered to the subject chronically, for a long period of time, for example for several months or even for several years, for the purposes of maintaining the value of muscle mass and/or of muscle function at a predetermined level, indicative of the absence of sarcopenia.

As shown in the examples, a composition in accordance with the present description, which comprises at least bacteria of the strain SGC-K, has insulin-sensitizing properties, i.e. has the ability to reduce insulin resistance in a subject, in particular in a diabetic subject or in a subject having a cardiovascular pathological condition, in particular a cardiovascular pathological condition linked to diabetes.

The description also relates to a composition as defined in the present application for use thereof for reducing insulin resistance in a subject in need thereof, in particular in a diabetic subject or in a subject having a cardiovascular pathological condition, in particular a cardiovascular pathological condition linked to diabetes.

The description relates to the use of a composition as defined in the present application for the manufacture of a medicament for reducing insulin resistance in a subject in need thereof, in particular in a diabetic subject, and more particularly a subject affected by type 2 diabetes, or in a subject having a cardiovascular pathological condition, in particular a cardiovascular pathological condition linked to diabetes.

The description also relates to a method for reducing insulin resistance in a subject in need thereof, in particular in a diabetic subject, comprising the administration to said subject of a composition as defined in the present application.

In general, a therapeutic composition according to the present description may be administered to the subject until the value of insulin resistance, for example until their HOMA-IR index, reaches a predetermined value, indicative of reduced insulin resistance.

In general, a therapeutic composition according to the present description may be administered to the subject until the value of muscle mass and function, for example until their HOMA-IR index, reaches a predetermined value, indicative of muscle mass and/or of muscle function of a healthy subject.

In certain embodiments, a therapeutic composition in accordance with the present description is administered to the subject chronically, for a long period of time, for example for several months or even for several years, for the purposes of maintaining the value of muscle mass and/or of muscle function, for example the HOMA-IR index, at a predetermined value, indicative of reduced insulin resistance.

### Examples

### Example 1: Effect of the Strain L. casei SGC-K on Aged Animal Models with a Restricted Food Intake

### A. Materials and Methods

### A.1. Experimental Model and Diet

The experimental model that was used is the male Wistar rat (Charles River, Écully), aged 18-20 months. The animals were reared during the experimental period in an inverted nycthemeral cycle (night from 8 am to 8 μm and day from 8 μm to 8 am). The animals were placed in individual cages two weeks before the start of the experiment (before the start of the restriction) and then put into batches (15 animals per batch) by homogenizing their weight, their fat mass and their lean body mass.

The aged rats (Wistar males, 18-20 months) were put on dietary restriction in order to mimic the frailty of the elderly subject generally associated with undernutrition. Since the protein intake of frail elderly people is estimated, for a majority of this population, at less than 80% of the recommendations (Sieber, 2019, Malnutrition and sarcopenia: Aging Clinical and Experimental Research, Vol. 31 (6): 793-798), the animals were therefore restricted to 75% of ad libitum in order to mimic a similar nutritional situation. The group of restricted animals (group R) were compared with a group that were able to eat at will (group AL) and with a group of restricted animals receiving the strain L. casei SGC-K probiotic (group R+L. casei SGC-K). The latter were supplemented daily with probiotic bacteria (˜5×10⁹ bacteria/day) for 1 month (30-day experiment) or 2 months (60-day experiment).

Since the level of feeding was controlled and similar in the restricted control animals which were or were not supplemented with probiotic L. casei SGC-K, the effect of the probiotic was compared relative to restricted control animals, and relative to animals with normal ingestion levels.

A.2. Supplementation with Probiotics

The probiotic bacteria L. casei SGC-K used were concentrated in tubes frozen at −20° C. The probiotic bacteria were given alive to the supplemented rats (group R+SGCK) every morning, before the daily meal, at a dose of 5×10⁹ bacteria per rat. The distribution of the probiotics (or of the control buffer without probiotic) was carried out before the daily meal in order to limit the stress which would be caused by the gavage.

The bacterial pellet was taken up in the phosphate-buffered saline (PBS), and dispersed at the desired dose of 5×10⁹ bacteria on food pellet powder (1 g). For the rats in the restricted group (R), an equivalent volume of PBS was deposited on the food pellet powder, in order to limit any experimental biases. The bacteria are ingested by the rats in 15 minutes, and then the rest of the daily food is provided. The survival of the bacteria diluted in the PBS and on the food pellets for 30 minutes had been verified beforehand in vitro.

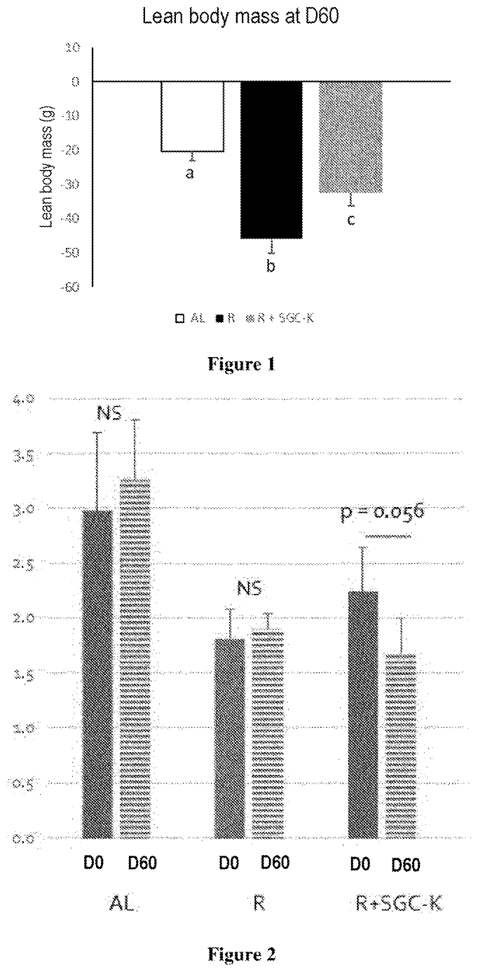

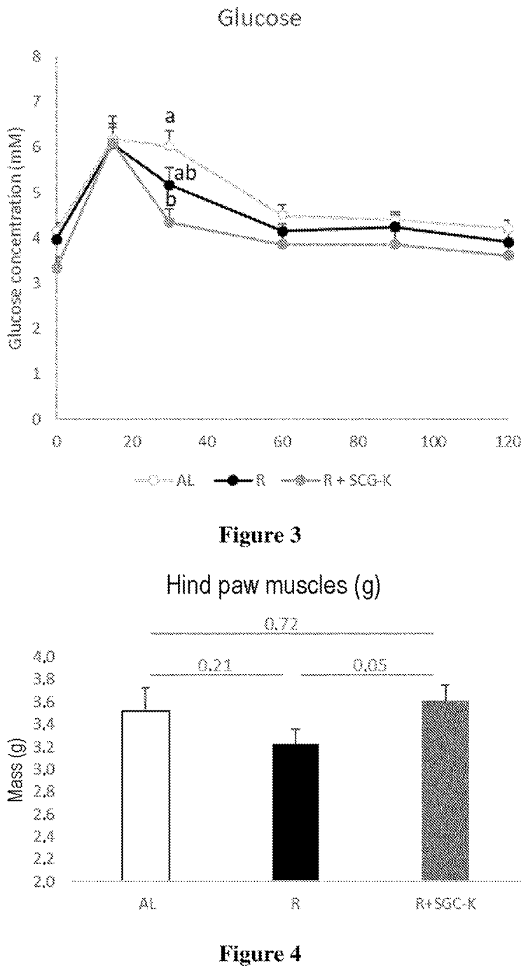

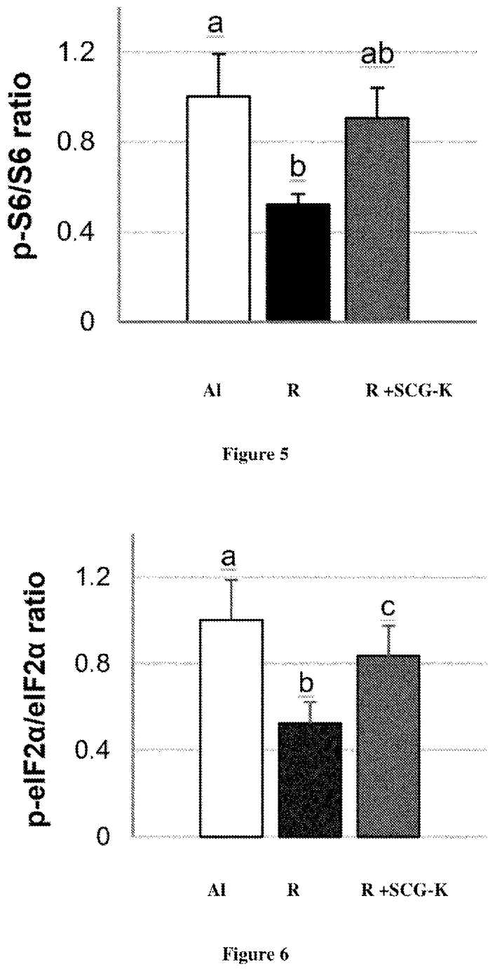

Two experiments were carried out: An experiment over 60 days made it possible to obtain longitudinal results relating to the lean body mass (FIG. 1) and the insulin sensitivity parameters (FIGS. 2 and 3). A shorter experiment lasting 30 days made it possible to generate endpoint data on the muscle mass (FIG. 4) and the muscle proteins (FIGS. 5 and 6).

### A.3. Longitudinal Phenotypic Monitoring

### A.3.1. Weight Monitoring

The animals were weighed three times per week throughout the duration of the experiment. This notably made it possible to monitor that the rats did not lose more than 20% of their initial weight (ethical criterion for animal testing), and to determine whether the taking of L. casei SGC-K probiotics had a specific effect on their weight.

### A.3.2. Body Composition Monitoring—60-Day Experiment

Body composition was evaluated by the EchoMRI method (Echo Medical Systems, Houston, TX USA). The measurements carried out by this device are based on the inherent differences in the nuclear magnetic resonance properties of hydrogen atoms and the relative density of hydrogen in fluids and tissues to obtain estimates of fat mass and lean body mass. The conscious rat is placed in a translucent plastic tube and immobilized. The holder is then inserted into a tubular space on the side of the EchoMRI system and the measurement is started. This allows a non-invasive analysis of body composition in less than three minutes on conscious animals. The body composition of the animals was measured at D7, at D30 and at D56.

A.3.3. Glucose Tolerance Test Before and After the Treatment with Probiotics-60-Day Experiment

This test consists in gavage of the fasted animals at a rate of 1 g of glucose per kg of rat's body mass and makes it possible to estimate the insulin and glycemic response to this glucose bolus, therefore the level of insulin sensitivity. A peripheral blood sample (tail vein) was taken while fasting. Then, blood samples are taken 15, 30, 60, 90 and 120 minutes after ingestion of the glucose bolus. The plasma is obtained after centrifugation at 1300 rpm for 10 minutes. The insulin was assayed in the plasma by ELISA (Mercodia rat insulin ELISA) and the glycemia by enzymatic assay (Glucose GOD-PAP, Biolab). The readings were taken using the Tecan Infinite 200 Pro UV/visible plate reader (Tecan, France). From the fasting glycemia and insulinemia, the HOMA (Homeostasis Model Assessment of insulin resistance) insulin resistance index can be calculated according to the following formula: (fasting insulinemia (mU/L)×fasting glycemia (mM))/22.5. The OGTTs were carried out before the ingestion of the probiotics and 2 days before the euthanasia of the animals at D60.

### A.4 Endpoint Effect on Muscle Mass—30-Day Experiment

This measurement consists in dissecting the main muscles of the hind paw (gastrocnemius, tibialis anterior, soleus and extensor digitorum longus) of the rats, weighing them and summing the masses at the end of the experimental period.

### A.5. Statistical Analyses

All analyses of statistical calculations were performed using the “Sigma Plot” software. The data are expressed as mean±standard error of the mean (SEM).

A two-way repeated measures analysis of variance (ANOVA) was used for the kinetic analysis of ingested data, of body mass monitoring, as well as for the data of the glucose tolerance tests (glycemia, insulinemia and HOMA). A one-way analysis of variance (ANOVA) was carried out for the analysis of the endpoint data of the fat mass and of the lean body mass (or of their evolution: value at D56-DO), the mass of the different tissues at euthanasia as well as the metabolite/hormone concentrations. The Holm-Sidak test was used for post-hoc comparisons of the means. Student's t-tests were carried out on the endpoint hind paw muscle weight data.

In all cases, the hypothesis of the existence of significant differences between the factors tested is validated for a risk of error of less than 5%. A trend is mentioned for a risk of error of 5% to 10%.

### B. Results

B.1. Weight and Body Composition (60-Day Experiment) (n=59)

The consumption of the animals was monitored throughout the experiment and shows that the R and R+SGC-K animals ingested on average respectively 21.3±0.1 g and 20.9±0.1 g of food while it was 28.9±0.3 g for the AL animals. The dietary restriction resulted in a decrease in body mass from the 7th day of restriction in comparison with the non-restricted animals (treatment effect P<0.001; R, R+SGC-K vs AL). The supplementation with L. casei SGC-K probiotics (group R+SGC-K) did not have any specific effect during the restriction. At the end of the study (60-day experiment), the mean loss of body mass of the R and R+SGC-K animals was 13.6±0.6%.

The other component of the loss of body mass is the loss of lean body mass. The loss of lean body mass is significantly greater with the restriction in the R and R+SGC-K animals in comparison with AL (P<0.05) (FIG. 1).

These results indicate that the ingestion of the probiotic L. casei SGC-K makes it possible to limit the loss of lean body mass which is caused by undernutrition.

B.2. Insulin Sensitivity State (60-Day Experiment) (n=53)

The insulin sensitivity state of the animals was evaluated at D7 and D58 using the HOMA-IR index. Between D7 and D58, the value of the HOMA-IR index (composite index integrating insulinemia and glycemia) does not vary significantly over time within each batch of animals (FIG. 2). When comparing the value of the HOMA-IR index between the different treatments at D58, it is observed that the dietary restriction tends to reduce the HOMA-IR for group R+SGC-K compared to AL (P=0.066; P=0.068 respectively), without being significant for R.

The results show that the excursion of the insulinemia is identical for all the animals with restricted nutrition, which includes (i) the group R of control animals with restricted nutrition and (ii) the group R+of animals with restricted nutrition having received the probiotic L. casei SGC-K.

When fasting, the value of the HOMA-IR index tends to decrease over the course of two months of supplementation with the probiotic L. casei SGC-K (group R+).

The analysis of the OGTT data at D58 shows that the glycemic excursion of group R+SGC-K was reduced with, 30 minutes after the ingestion of the glucose bolus, a glycemia lower than the AL batch (P=0.015) (FIG. 3).

In addition, the results of FIG. 3 show that the glycemia of the animals in group R+SGC-K having received the probiotic L. casei SGC-K decreases significantly more rapidly, 30 minutes after the ingestion of the bolus, in comparison with the animals in group R also with restricted nutrition but which have not received probiotic (P=0.01) (FIG. 3).

All of these results suggest that the probiotic L. casei SGC-K causes an insulin-sensitizing effect.

The insulin-sensitizing effect which is caused by the ingestion of the probiotic L. casei SGC-K could contribute to the preservation of lean muscle mass.

B.3 Effect on Muscle Mass (30-Day Experiment) (n=61)

These results have been obtained from a different study (over 30 days) than that which allowed the longitudinal monitoring of the evolution of lean body mass and of insulin sensitivity (the latter carried out over 60 days). The mass of the four muscles of a hind paw of rats (gastrocnemius, tibialis anterior, soleus and extensor digitorum longus) was measured at the end of a 30-day experimental period. The results show that the probiotic L. casei SGC-K made it possible to maintain the muscle mass of undernourished rats (group R+SGC-K and AL not different, P=0.72) with, in parallel, a significantly higher muscle mass in the rats of group R+SGC-K relative to group R (+12%, P=0.05).

These data suggest that the ingestion of the probiotic L. casei SGC-K made possible the preservation of muscle mass, even in a context of undernutrition.

### Example 2: Effect of the Strain L. casei SGC-K on Insulin Sensitivity in Aged Animal Models with a Restricted Food Intake

### A. Materials and Methods

The experimental animal models were carried out as described in Example 1.

A. Materials and Methods of the Western Blots (30-Day Experiment) (n=61)

The Western blot enables the detection and relative quantification of the phosphorylated (pS6 and p-eIF2α) or non-phosphorylated (S6 and eIF2α) proteins S6 and eIF2α. The phosphorylated/dephosphorylated protein quantity ratio of S6 and eIF2a illustrates the level of activation of the mTOR signaling pathway. The gastrocnemius muscles (50 mg) of the animals (30-day protocol) were frozen in liquid nitrogen and ground to reduce them to powder. The samples were then homogenized in the extraction buffer, centrifuged (10 000 g at 4° C. 10 min) to recover the supernatants. The supernatants are diluted and kept at −20° C. to determine the protein concentration (Pierce™ BCA Protein Assay Kit). 16 μg of proteins are loaded onto a denaturing SDS-PAGE gel (Mini-PROTEAN TGX Precast Gels, Bio-Rad, France) and transferred onto a PVDF membrane (Trans-Blot® Turbo™ Midi-size PVDF, Bio-Rad, France). The membrane was prehybridized with 5% of BSA (bovine serum albumin). The primary antibodies are rabbit p-S6, S235/236 (Cell Signaling Technology 2211S), rabbit S6 (Cell Signaling Technology 2217S), rabbit p-eIF2a S51 (abcam 32157), rabbit eIF2a (abcam 242148) diluted 1:1000 or 1:2000. The secondary antibodies are anti-rabbit HRP-linked antibodies (Cell Signaling Technology 7074) diluted 1:2000. After prehybridization, hybridization and washings, the signals of the bands were quantified with the GeneTools software (Syngene).

### B. Results

As in the case of the whole organism, it is shown in Example 2 that proteins involved in the intracellular signaling of insulin, such as the protein S6 and the elongation factor elF2a, were stimulated by the bacterial strain SGCK (in a postprandial situation) in the muscle in the model of frail aged rats (in chronic undernutrition).

After the ingestion of the meal, (i) an intracellular mediator of insulin action, namely the phosphorylation of the protein S6, represented in FIG. 5, and (ii) the phosphorylation of the elongation factor elF2a involved in the stimulation of protein synthesis, also sensitive to insulin, represented in FIG. 6, were measured.

The results represented in each of FIGS. 5 and 6 show that these two indicators of insulin sensitivity were less stimulated in the animals in the adapted situation of restriction (R) relative to the animals fed ad libitum (T). The supplementation with strain SGCK enabled these two proteins to exhibit a level of phosphorylation close to that of the control animals (p-S6/S6) or intermediate between T and R (p-eIF2a/eIF2a).

The results presented in FIGS. 5 and 6 show an improvement in the muscle insulin sensitivity in the animals supplemented with probiotic SGCK. Furthermore, these mediators are involved in the stimulation of protein synthesis linked to the ingestion of the meal. This mechanism contributes to the preservation of muscle mass observed in rats supplemented with the strain SGCK.

### Reference to the Deposited Biological Material

Bacterial strain of L. casei SGC-K deposited on Mar. 8, 2021 with the Collection Nationale de Cultures de Microorganismes [French National Collection of Microorganism Cultures] of the Institut Pasteur, 25 rue du Docteur Roux—75724 Paris Cedex 15 (France), under No. CNCM I-5663.

1. A composition comprising bacteria of the strain L. casei SGC-K deposited with the Collection Nationale de Cultures de Microorganismes [French National Collection of Microorganism Cultures] of the Institut Pasteur on Mar. 8, 2021 under No. CNCM 1-5663.

2. The composition as claimed in claim 1, wherein the bacteria of the strain L. casei SGC-K are combined with the bacteria of one or more other probiotic bacterial strains.

3. The composition as claimed in claim 2, the other probiotic bacterial strains being chosen from the genera Bifidobacterium, Lactobacillus, Lactococcus, Enterococcus, Streptococcus, Kluyveromyces, Saccharomyces, Faecalibacterium, Blautia, Christensenella, Bacteroides, Eubacterium, Roseburia, Coprococcus and combinations thereof.

4. The composition as claimed in claim 2, the other probiotic bacterial strains being chosen from the species Bifidobacterium longum, Bifidobacterium lactis, Bifidobacterium breve, Bifidobacterium infantis, Bifidobacterium adolescentis, Lactobacillus acidophilus, Lactobacillus casei, Lactobacillus paracasei, Lactobacillus salivarius, Lactobacillus lactis, Lactobacillus rhamnosus, Lactobacillus johnsonii, Lactobacillus plantarum, Lactobacillus salivarius, Lactococcus lactis, Enterococcus faecium, Enterococcus faecalis, Saccharomyces cerevisiae, Saccharomyces boulardii, Faecalibacterium prausnitzii or mixtures thereof, preferably chosen from the group consisting of Bifidobacterium longum NCC3001 (ATCC BAA-999), Bifidobacterium longum NCC2705 (CNCM 1-2618), Bifidobacterium longum NCC490 (CNCM 1-2170), Bifidobacterium lactis NCC2818 (CNCM I-3446), Bifidobacterium breve strain A, Lactobacillus paracasei NCC2461 (CNCM 1-2116), Lactobacillus johnsonii NCC533 (CNCM 1-1225), Lactobacillus rhamnosus GG (ATCC53103), Lactobacillus rhamnosus NCC4007 (CGMCC 1. 3724), Enterococcus faecium SF 68 (NCC2768; NCIMB10415), and combinations thereof.

5. The composition as claimed in claim 1 further comprising one or more prebiotics.

6. A method for maintaining or increasing muscle mass and/or function in a subject in need thereof, in particular in a malnourished subject, in an elderly subject, in particular a malnourished elderly subject, and in a subject performing intense physical exercise comprising administering to the subject the composition of claim 1.

7. (canceled)

8. A method for the treatment of the loss of muscle mass and/or function in a subject in need thereof, in particular in a subject chosen from overweight subjects and obese subjects subjected to a diet, cancer subjects affected by sarcopenia, diabetic subjects subjected to a diet comprising administering to the subject the composition of claim 1.

9. A method for reducing insulin resistance in a subject in need thereof, in particular in a diabetic subject, and more particularly a subject affected by type 2 diabetes comprising administering to the subject the composition of claim 1.

10. A method for the prevention or the treatment of inflammatory pathological conditions chosen from (i) irritable bowel syndrome or disease, inflammatory bowel diseases, (ii) chronic obstructive pulmonary disease, or else (iii) other pathological conditions linked to chronic inflammation, including chronic intestinal or kidney inflammation, such as long-term inflammation caused by a viral infection such as an infection by a SARS-COV-2 virus comprising administering to a subject in need thereof the composition of claim 1.

11. A strain L. casei SGC-K deposited with the Collection Nationale de Cultures de Microorganismes [French National Collection of Microorganism Cultures] of the Institut Pasteur on Mar. 8, 2021 under No. CNCM I-5663.

12. The composition as claimed in claim 3, the other probiotic bacterial strains being chosen from the species Bifidobacterium longum, Bifidobacterium lactis, Bifidobacterium breve, Bifidobacterium infantis, Bifidobacterium adolescentis, Lactobacillus acidophilus, Lactobacillus casei, Lactobacillus paracasei, Lactobacillus salivarius, Lactobacillus lactis, Lactobacillus rhamnosus, Lactobacillus johnsonii, Lactobacillus plantarum, Lactobacillus salivarius, Lactococcus lactis, Enterococcus faecium, Enterococcus faecalis, Saccharomyces cerevisiae, Saccharomyces boulardii, Faecalibacterium prausnitzii or mixtures thereof, preferably chosen from the group consisting of Bifidobacterium longum NCC3001 (ATCC BAA-999), Bifidobacterium longum NCC2705 (CNCM 1-2618), Bifidobacterium longum NCC490 (CNCM 1-2170), Bifidobacterium lactis NCC2818 (CNCM I-3446), Bifidobacterium breve strain A, Lactobacillus paracasei NCC2461 (CNCM 1-2116), Lactobacillus johnsonii NCC533 (CNCM 1-1225), Lactobacillus rhamnosus GG (ATCC53103), Lactobacillus rhamnosus NCC4007 (CGMCC 1. 3724), Enterococcus faecium SF 68 (NCC2768; NCIMB10415), and combinations thereof.

13. The composition as claimed in claim 2 further comprising one or more prebiotics.

14. The composition as claimed in claim 3 further comprising one or more prebiotics.
